(19) United States
(12) Patent Application Publication
(10) Pub. No.: US 2026/0306856 A1
(43) Pub. Date: Oct. 8, 2026

(54) A WEARABLE ELECTRONIC DEVICE COMPRISING AN RF ANTENNA

(71) Applicant: UNEEG MEDICAL A/S, Alleroed (DK)

(72) Inventors: Per HILLERSBORG, Virum (DK); Jacob Geert JENSEN, Copenhagen (DK)

(73) Assignee: UNEEG MEDICAL A/S, Alleroed (DK)

(21) Appl. No.: 19/481,757

(22) PCT Filed: May 7, 2024
(86) PCT No.: PCT/EP2024/062574

§ 371 Date: Nov. 5, 2025

(30) Foreign Application Priority Data
May 10, 2023 (DK) PA202330022

(51) Int. Cl. A61N 1/372; H01Q 1/27; H01Q 1/38; H01Q 9/42; H04R 1/10; H04R 25/00

(52) U.S. Cl. CPC A61N 1/37229; H01Q 1/273; H01Q 1/38; H01Q 9/42; H04R 1/1016; H04R 25/609; H04R 25/652; H04R 2225/51

(57) ABSTRACT

Please add the following new Abstract of the Disclosure:

A wearable electronic device is provided which may be, e.g., one of an EEG logger, a hearing aid and a cochlear implant. The wearable electronic device includes electronics mounted on a printed circuit board and an RF antenna. The RF antenna is formed by two surface conductors, i.e. an antenna trace and a ground plane, being arranged with a distance parallel with respect to each other or inclined towards each other at a small angle. The printed circuit board, in a direction perpendicular to the ground plane, is arranged between the antenna trace and the ground plane, with a distance to each of the antenna trace and the ground plane.

The invention relates to a wearable electronic device comprising electronics mounted on a printed circuit board (PCB), and an RF antenna. In preferred embodiments, the wearable electronic device is a wearable medical device, e.g. a monitoring device such as an EEG logger, a hearing aid, a cochlear implant or an insulin pump. However, the invention is also applied with benefit to a large variety of non-medical wearable electronic devices, such as in-ear head phones (ear plugs), wrist watches, smart glasses, etc.

Wearable electronics devices (so-called “wearables”, i.e. small electronic devices designed to be worn at the body of a user) such as EEG loggers are often designed to be connected wirelessly to at least one further electronic device such as, for example, a smart phone or a tablet computer. Typically, the further electronic device is used as a host (i.e. a resource for computing power and data storage) of a software application attributed to the wearable. This software application (referred to as the “service app”) may be used for remote control, configuration and/or update of the wearable or as an external data storage to the latter. Nowadays, the wireless connection between the wearable and the further electronic device is often based on the Bluetooth standard, using RF fields in the GHz (Giga-Hertz) regime of the electromagnetic spectrum.

In order to send and/or receive RF signals, a wearable typically comprises an RF antenna. Often, a planar design is chosen for the RF antenna since planar antennas (“patch antennas”) can be realized in a compact (i.e. space-saving) form and can be easily integrated with a printed circuit board (PCB) carrying the electronics of the wearable. In a wearable that is worn at the user's head, such as an EEG logger, a planar design of the RF antenna is also preferred as it can be easily designed to create an electro-magnetic field with a polarisation of the electric field being perpendicular to the head, which is beneficial in order to achieve a good crossbody performance.

A planar RF antenna comprises two planar conductors arranged parallel and spaced with respect to each other, namely an antenna patch and a ground plane. When integrated with the PCB, typically, the ground plane is formed as a planar metallized structure which is directly applied to one side of the PCB. In some applications, the antenna patch may be formed as a conductive structure on the opposite side of the PCB such that the antenna patch and the ground plane are separated by a substrate of the PCB. However, in order to achieve a sufficient bandwidth, in particular for a wireless connection based on the Bluetooth standard, the antenna patch and the ground plane should be arranged in a distance (of, e.g., ca. 2.5 mm or more) that exceeds the thickness of a normal PCB. Therefore, the antenna patch is often realized separately from the PCB and arranged in a distance to the latter, e.g. as a sheet-metal part.

In some wearables having a very flat housing shape such as modern EEG loggers, arrangement of the PCB and the RF antenna is a problem, even if the RF antenna is realized as a conventional planar antenna, for lack of space for locating the antenna patch in a sufficient distance to the PCB.

US 2016/285157 A1 discloses a wearable electronic device, in particular a smart watch. The device comprises electronics mounted on a printed circuit board, and an RF antenna. The RF antenna comprises two surface conductors being arranged with a distance and parallel with respect to each other, said two surface conductors being an antenna trace and a ground plane, wherein the ground plane is arranged on the printed circuit board. The RF antenna further comprises a second radiator that is arranged opposite to the antenna trace, also with a distance to the printed circuit board and the ground plane arranged thereon.

An object of the present invention is to provide a way of arranging a PCB and an RF antenna of a wearable electronic device in a very compact, in particular flat, manner.

According to the invention the above object is met by a wearable electronic device (subsequently referred to as a “wearable”) as defined by claim 1. Preferred embodiments of the invention are described in the dependent claims and the subsequent description.

The wearable comprises electronics mounted on a printed circuit board (PCB), and an RF antenna. Preferably, the RF antenna has an at least approximately planar antenna design. It is formed by two surface conductors (i.e. two-dimensionally extended and, preferably, at least approximately planar conductors) being arranged with a distance and parallel with respect to each other or inclined towards each other at a small angle, said two surface conductors being an antenna trace (which may also be referred to as “antenna strip” or “antenna patch”) and a ground plane. In a direction perpendicular to the ground plane, the PCB is arranged between the two surface conductors of the RF antenna (i.e. the antenna trace and the ground plane), with a distance to each of the antenna trace and the ground plane. As seen in said perpendicular direction, the PCB may be arranged so to overlap with at least one of the surface conductors, in particular with the ground plane. However, in an alternative arrangement consistent with the invention, the PCB may be shifted with respect to the surface conductors in a lateral direction (parallel to the surface in which the ground plane extends) such that the PCB does not overlap with the surface conductors when seen in said perpendicular direction. Preferably, the PCB is arranged at least approximately parallel with respect to at least one of the surface conductors, in particular with respect to the ground plane.

In embodiments in which the antenna trace and the ground plane are inclined towards each other at a small angle, preferably, said small angle between the antenna trace and the ground plane is ≤25° (preferably ≤20°, e.g. ca. 16.5°). Also, at least one of the respective surfaces of the antenna trace and the ground plane may have a slight curvature such that at least a part of this surface may be inclined with respect to the other surface or a part thereof at a small angle as defined above. Preferably, at least one of the antenna trace and the ground plane are designed conformal with the housing (i.e. have a surface the shape of which is adapted to the shape of an adjacent portion of the housing wall).

The term “at least approximately planar” means that each of the respective surfaces of the antenna trace and the ground plane may be exactly planar (i.e. flat) or have a slight overall curvature with a mean curvature radius being at least 4 times larger than a maximum lateral extension of the respective conductor. In an example, the antenna trace qualifies as “at least approximately planar” if it has a mean curvature radius of ca. 4 mm and a maximum lateral extension (width) of ca. 0.8 mm such that its mean curvature radius is ca. 5 times larger than its maximum lateral extension. In another example, the ground plane qualifies as “at least approximately planar” if it has a rounded border area next to a planar centre region such that its mean curvature radius still is more than 4 times larger than its maximum lateral extension.

Within the scope of the invention, at least one of the two surface conductors may be designed as a self-supporting structure (e.g. from a stamped metal sheet) or be applied to a supporting structure, different from the PCB and independently of the second surface conductor. In particular, at least one of the two surface conductors may be applied on an inside wall portion of the housing which, in this case, acts as a supporting structure for the surface conductor.

However, in a preferred embodiment of the invention, the two surface conductors are applied on opposite sides of a non-conducting supporting part, separate and different from the PCB and the housing, which is subsequently referred to as the “carrier”. Preferably, the carrier is made of plastic, in particular made by injection moulding.

The invention is based on the idea of off-setting the ground plane of the RF antenna with respect to the PCB, e.g. by using the separate carrier, and placing the PCB between the two surface conductors. Hereby, on the one hand, the full height of the inner space of a housing of the wearable can be used for arranging the two surface conductors in a sufficient distance whereas, on the other hand, the PCB can be arranged independently of the RF antenna, taking into account the space requirements of electronic units mounted on the PCB. As a result, the invention allows for or, at least, simplifies designing wearables with a very flat housing without interfering with requirements or desired properties of the RF antenna and the PCB.

In an embodiment of the invention, the carrier is attached to the printed circuit board by at least one soldered joint. Preferably, said at least one soldered joint has a double function serving both as a means for mechanically fixing the carrier to the PCB and a means for electrically (conductively) connecting at least one of the surface conductors with the PCB. In a particular embodiment, the carrier is attached to the printed circuit board by at least two separate soldered joints that electrically connect both the antenna trace and the ground plane to the PCB. In further embodiments of the invention, different connection means for electrically and/or mechanically connecting the carrier to the PCB may be used, such as C-springs, EMC gaskets, etc.

In an embodiment of the invention, the carrier has an L-shaped or C-shaped cross-section. In the first case, the L-shaped carrier (as seen in cross-section) has a first leg being oriented at least approximately parallel to the PCB and a second leg being oriented at least approximately perpendicular with respect to the PCB. In the latter case, the C-shaped carrier (as seen in cross-section) has two legs extending at least approximately parallel to the PCB and framing a hollow groove between them. In both cases, preferably, at least one of said legs overlaps with the PCB, e.g. for attaching the carrier to the PCB and/or for arranging at least one of the surface conductors so to overlap with the PCB.

In a preferred embodiment of the invention, the carrier has curved outer contour at a side facing away from the printed circuit board. This simplifies arrangement of the RF antenna in a housing of the wearable having a rounded shape (e.g. cylindrical, oval, or polygonal with rounded edges) while ensuring efficient utilization of the space available for arrangement of the surface conductors.

In advantageous embodiments of the invention, the RF antenna is formed as one of an inverted L antenna, and an inverted F antenna.

Preferably, the wearable is a medical device, in particular an EEG logger, a hearing aid or a cochlear implant. However, in further embodiments, the wearable may also be also be a non-medical consumer product such as, e.g., an in-ear head phone (ear plug), a wrist watch (smart watch) or a pair of smart glasses.

Subsequently, embodiments of the present invention will be described with reference to the accompanying drawings in which

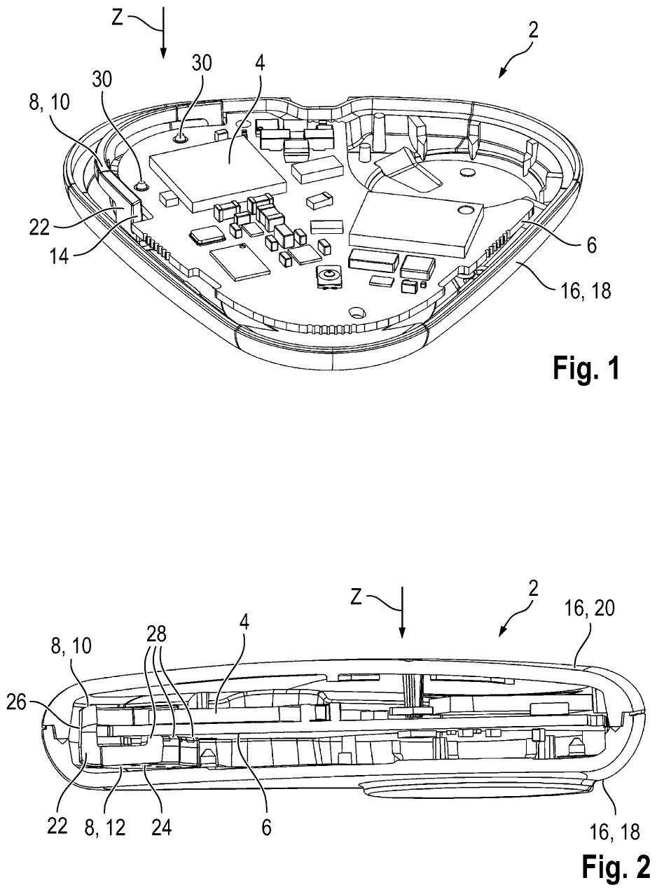

FIG. 1 shows a perspective view on a top side of an EEG logger as an example of a wearable electronic device, the EEG logger comprising a housing (shown without atop shell of said housing) in which electronics mounted on a printed circuit board (PCB), and an RF antenna are arranged, wherein the RF antenna is formed by two surface conductors, i.e. an antenna trace and a ground plane, wherein the two surface conductors are applied on opposite sides of a carrier;

FIG. 2 shows a cross-sectional view through the housing, the PCB and carrier of the EEG logger of FIG. 1;

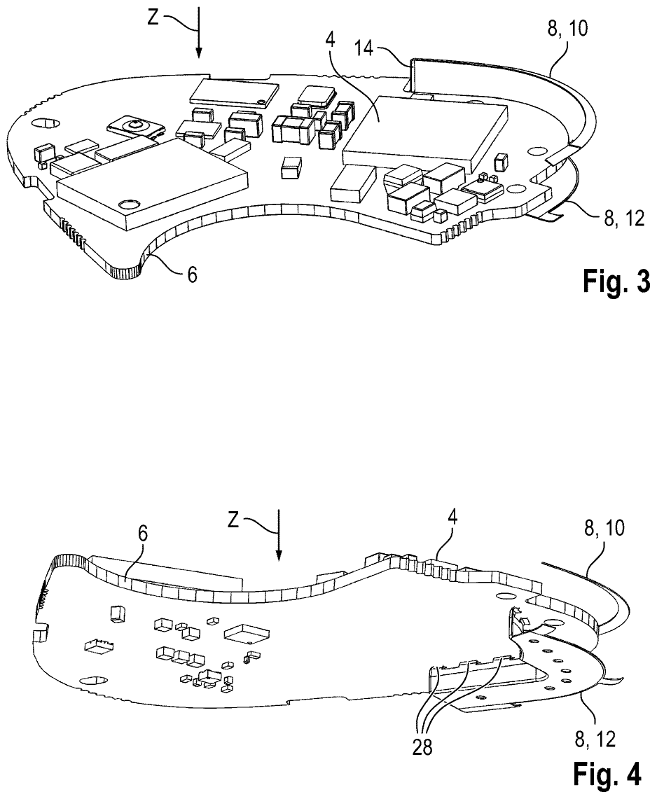

FIG. 3 shows a perspective view on a top side of an arrangement comprising the PCB and two surface conductors of the surface RF antenna as mounted in the housing (however without the housing and the carrier); and

FIG. 4 show a perspective view on a bottom side of the arrangement of FIG. 3.

Like reference numerals indicate like parts, structures and elements unless indicated other-wise.

The FIGS. 1 to 4 show a first embodiment of an EEG logger 2, i.e. a wearable electronic device for monitoring electroencephalogram (EEG) data, i.e. bio-electric brain waves of a human user (patient). The EEG logger 2 wirelessly communicates, e.g. using near field induction communication, with an EEG sensor (not shown) which is implanted subcutaneously in the head of the user. The EEG logger 2 is configured to be worn at the head of the user, in close vicinity to the sensor. For instance, the EEG logger 2 may be designed to be removably fixed in a holder (also referred to as the “base plate”) that, in turn, is adhesively fixed at the scalp of the user.

In operation, the EEG logger 2 triggers the sensor to measure EEG data and receives the EEG data measured and sent to the EEG logger 2 by the sensor. The EGG logger 2 is configured to store the received EEG data for later evaluation by a health care professional, transfer the received EEG data wirelessly to a remote evaluation unit and/or evaluate the received EEG data in real time, e.g. in order to detect imminent or otherwise unrecognized epileptic seizures.

These and other functions of the EEG logger 2 are implemented by electronic units (referred to as electronics 4) which are mounted on a printed circuit board (PCB 6) of the EEG logger 2. By preference, the electronics 4 comprise at least one programmable unit such as a microcontroller. In this case, at least some of the functions of the EEG logger 2 are implemented as software installed in and executable by the programmable unit.

Furthermore, the EEG logger 2 comprises an RF antenna 8 enabling the EEG logger 2 to communicate wirelessly with a further electronic device (not shown) such as a smart phone or tablet computer of the user, based on the Bluetooth standard. In preferred embodiments, the EEG logger 2 uses the RF antenna 8 to interact with a software application (referred to as the service app) that is installed on the further electronic device and configured to remote control, configure and/or update the EEG logger 2. Preferably, the EEG logger 2 also uses the RF antenna 8 to send EEG data, status messages and/or alerts to the service app which may display at least part of this data to the user and/or relay it to a remote health care server, e.g. via a wireless connection to the internet.

The RF antenna 8 is formed by two—at least approximately planar—surface conductors, i.e. an elongate and thin antenna trace 10 and an elongate but (as to compared to the antenna trace 10) significantly broader ground plane 12. The antenna trace 10 and the ground plane 12 are arranged so to extend approximately parallel to each other with a distance of, e.g., ca. 3 mm. In detail, the antenna trace 12 and the ground plane 10 are inclined towards each other at a small angle of ca. 16.5°. The ground plane 12 is approximately flat with a curved outer edge portion that is adapted to the curvature of an adjacent housing portion. The antenna trace 10 has a slightly curved surface with a curvature radius (e.g. of 4.14 mm) that is about 5 times larger that its width (e.g. of 0,8 mm).

The antenna trace 10 and the ground plane 12 may be aligned with each other such that, as seen in a direction Z perpendicular to the ground plane 12, the antenna trace 10 may completely overlap with the ground plane 12. However, as can be seen in FIGS. 3 and 4 for the exemplary embodiment, the antenna trace 10 may also be shifted with respect to the ground plane 12 in a lateral direction (perpendicular to the direction Z) such that there is little or even no overlap of the antenna trace 10 and the ground plane 12, as seen in the direction Z.

In the example of FIGS. 1 to 4, the RF antenna 8 is designed as a (at least approximately planar) Inverted L antenna; i.e. the antenna trace 10 is connected to an antenna feed tab 14 at a first one of its longitudinal ends while the second longitudinal end of the antenna trace 10 is isolated.

As can be seen from FIGS. 1 and 2, the PCB 6 and the RF antenna 8 are arranged in a housing 16 which is formed of two parts, i.e. a bottom shell 18 and a top shell 20. The housing 16, in a plan view to the bottom shell 18 or a top shell 20, has a flat triangular shape with rounded edges (cf. FIG. 1). It has a rounded cross-sectional contour (cf. FIG. 2). The EEG logger 2 is designed to be worn at a side of the user's head such that the bottom shell 18 is arranged adjacent to the head whereas the top shell 20 faces away from the head.

In view of a good wearing comfort of the EEG logger 2, i.e. in order to avoid that the user perceives the EEG logger 2 as annoying when it is worn at the user's head, the housing 16 is designed with a very flat shape. Preferably, its outer contour has a maximum thickness of only 5 to 8 mm. The inner space of the housing 16 has a thickness of ca. 3,5 to 5 mm only.

In order to arrange both the PCB 6 and the RF antenna 8 in the housing 16 while preserving sufficient space for the electronics 4 and a sufficient distance of the antenna trace 10 from the ground plane 12, the antenna trace 10 and the ground plane 12 are not integrated with the PCB 6. Instead, the antenna trace 10 and the ground plane 12 are formed as metallized layers being applied to opposite sides of a carrier 22 which is an injection moulded plastic part being manufactured separately from the PCB 6. Likewise, the antenna feeding tab 14 is formed as a conductive path applied to a surface of the carrier 22.

As can be seen in FIG. 2, the carrier 22 of the exemplary embodiment of the EEG logger 2 has an L-shaped cross-section. In the cross-sectional view, it has a first leg 24 that is oriented at least approximately parallel to the PCB 6, and a second leg 26 that is oriented at least approximately perpendicular with respect to the PCB 6. The first leg 24 carries the ground plane 12 at its outer surface, whereas its inner surface is attached to a bottom surface of the PCB 6. Both the first leg 24 and the ground plane 12 overlap with the PCB 6, as seen in the direction Z. The second leg 26 of the carrier 22 reaches around an edge of the PCB 6, thus protruding over a top surface of the PCB 6. At its free longitudinal end face, the second leg 26 carries the antenna trace 10. The carrier 22, thus, extends over almost the entire width of the inner space of the housing 16, with the ground plane 12 being arranged very close to the wall of the bottom shell 18 and the antenna trace 10 being arranged very close to the wall of the top shell 20. The PCB 6, on the other hand, is arranged in a centre of the inner space of the housing 16 being equally spaced, at least approximately, from both the bottom shell 18 and the top shell 20. It is, thus, arranged between the antenna trace 10 and the ground plane 12, as seen in the perpendicular direction Z, with a distance to both surface conductors. The ground plane 12 extends approximately parallel with respect to the PCB 6. In fact, the ground plane 12 is inclined towards the PCB 6 at a very small angle of, e.g. 1.8°, thus adapting to the shape of the bottom shell 18.

The carrier 22 is attached to the PCB 6 by several soldered joints 28. At the one hand, the soldered joints 28 are used to mechanically fix the carrier 22 to the PCB 6. On the other hand, the soldered joints 28 are used to electrically connect the antenna trace 10 (via the antenna feed tab 14) and the ground plane 12 to the PCB 6. The carrier 22 and the PCB 6 are positioned and fixed relative to each other by two pilot pins 30.

As can be seen in FIG. 1, the carrier 22 and, thus, the antenna trace 10 and the ground plane 12 are located in one of the rounded edges of the housing 16. Herein, in order to adapt the carrier 22 to the shape of the housing 16, the carrier 22—at its side facing away from the PCB 6—is made with a rounded outer contour that nestles against the inner side of the wall of the housing 16. In accordance with the shape of the carrier 22, the antenna trace 10 and the ground plane 12 have a curved shape.

In a second embodiment (not shown) of the EEG logger 2, different from the first embodiment shown in FIGS. 1 to 4, the RF antenna 8 is designed as a (at least approximately planar) inverted F antenna. In this case, the first longitudinal end of the antenna trace 10 is shorted with the ground plane (instead of being connected to the antenna feed tab 14). In this embodiment, the antenna feed tab 14 is connected to the antenna trace 10 at a feed point that is situated between and with a distance to both longitudinal ends of the antenna trace 10, wherein—preferably—the feed point is located nearer to the shorted first longitudinal end of the antenna trace 10 than to the isolated second longitudinal end.

In further embodiments, the wearable electronic device may be configured as a different medical monitoring device, e.g. a device for monitoring Electrocardiogram (ECG) or Electromyogram (EMG) data, pulse frequency, oxygen saturation of the blood, breath frequency or level of blood sugar. Furthermore, the wearable electronic device may be configured as a different medical device such as hearing aid, a cochlear implant or an insulin pump. The wearable electronic device may also be configured for a non-medical purpose, e.g. as an in-ear head-phone (ear plug), a smart watch or a pair of smart glasses.

It will be appreciated by persons skilled in the art that numerous variations and/or modifications may be made to the invention as shown in the specific examples without departing from the spirit and scope of the invention as broadly described in the claims. The present examples are, therefore, to be considered in all aspects as illustrative and not restrictive.

### LIST OF REFERENCE NUMERALS

2 EEG logger
4 electronics
6 PCB
8 RF antenna
10 antenna trace
12 ground plane
14 antenna feed tab
16 housing
18 bottom shell
20 top shell
22 carrier
24 leg
26 leg
28 soldered joint
30 pilot pin
Z direction

1. A wearable electronic device, in particular an EEG logger, a hearing aid or a cochlear implant, comprising electronics mounted on a printed circuit board and an RF antenna wherein the RF antenna is formed by two surface conductors being arranged with a distance and parallel with respect to each other or inclined towards each other at a small angle, said two surface conductors being an antenna trace and a ground plane, and wherein the printed circuit board, in a direction perpendicular to the ground plane, is arranged between the antenna trace and the ground plane, and with a distance to each of the antenna trace and the ground plane.

2. The wearable electronic device according to claim 1, wherein the two surface conductors are applied on opposite sides of a non-conducting carrier.

3. The wearable electronic device according to claim 1, wherein the printed circuit board is arranged at least approximately parallel to the ground plane.

4. The wearable electronic device according to claim 1, wherein the carrier is attached to the printed circuit board by at least one soldered joint.

5. The wearable electronic device according to claim 1, wherein the carrier has L-shaped or C-shaped cross-section having at least one leg that overlaps with the printed circuit board.

6. The wearable electronic device according to claim 1, wherein at least one of the two surface conductors, preferably at least the ground plane, overlaps with the printed circuit board, as seen in a direction perpendicular to the ground plane.

7. The wearable electronic device according to claim 1, wherein the carrier, at a side facing away from the printed circuit board, has curved outer contour.

8. The wearable electronic device according to claim 1, wherein the RF antenna is formed as one of an inverted L antenna, and an inverted F antenna.
